(19) United States
(12) Patent Application Publication
(10) Pub. No.: US 2026/0234341 A1
(43) Pub. Date: Aug. 13, 2026

(54) Method for Controlling the Crosslinking Time for a Silicone Gel That Gives Off a Volatile Organic Substance

(71) Applicant: V. MANE FILS, LE BAR-SUR-LOUP (FR)

(72) Inventors: André PEDRO, LE BAR-SUR-LOUP (FR); Elsa RAYNAUD, ROQUEFORT-LES-PINS (FR)

(21) Appl. No.: 19/141,583

(22) PCT Filed: Dec. 22, 2023
(86) PCT No.: PCT/FR2023/052110

§ 371 Date: Jun. 20, 2025

(30) Foreign Application Priority Data
Dec. 22, 2022 (FR) FR2214343

(51) Int. Cl. C08G 77/26; A61L 9/012; A61L 9/015; C08G 77/08; C08G 77/16; C11D 3/37; C11D 3/50

(52) U.S. Cl. CPC C08G 77/26; A61L 9/012; A61L 9/015; C08G 77/08; C08G 77/16; C11D 3/3742; C11D 3/505

(57) ABSTRACT

The present invention relates to a method for preparing a silicone gel that gives off a volatile organic substance, the method comprising the following steps: 1) providing a part A comprising (i) from 10% to 89.99% by weight of a dihydroxy PDMS polymer that has a viscosity measured at 25° C. of between 3000 and 100,000 cSt (mm²/s) and (ii) from 10% to 90% by weight of a mixture including a volatile solvent capable of solubilising the dihydroxy polydimethylsiloxane polymer and a volatile organic substance; 2) providing a part B comprising (iii) from 10% to 30% by weight of MMT and (iv) from 70% to 90% by weight of a non-functionalised polydimethylsiloxane silicone oil that has a viscosity measured at 25° C. of between 10 and 245 cSt (mm²/s) capable of solubilising N-morpholinomethyl triethoxysilane; 3) preparing a mixture comprising from 95% to 99% by weight of part A and from 1% to 5% by weight of part B; characterised in that part A also comprises from 0.01% to 5% by weight of a carboxylic acid and does not comprise a metal catalyst, and a gel that can be obtained by this method and the kit for implementing this method.

### TECHNICAL FIELD

The present invention relates to the field of articles used for fragrancing or deodorizing the atmosphere or laundry, or as insect repellents or insecticides, which comprise a solid polymer material containing a volatile organic substance. More particularly, the present invention relates to a transparent anhydrous silicone gel which diffuses volatile organic substances, said gel including neither a reinforcing agent nor a toxic catalyst and having the advantage of having a readily controllable crosslinking time.

### PRIOR ART

Numerous types of devices exist for releasing and/or diffusing volatile organic substances such as fragrances, odor-absorbing molecules or insecticides into the environment. These devices can be in various forms suitable for their uses and can be obtained in various ways. Generally, these products consist of an organic matrix (notably polymeric) of the silicone type.

Various technologies currently exist for producing this type of organic polymer matrix, notably matrices obtained from silicone elastomers in which one or more volatile organic substances are dispersed.

A preferred type of silicone elastomer is cold-vulcanizable silicone elastomers (CVE) formulated from reactive polydimethylsiloxanes (PDMS) with varying degrees of polymerization. Crosslinking takes place at room temperature by virtue of a crosslinking agent which reacts with the reactive groups in the polysiloxane chains, and a catalyst which allows the crosslinking to be controlled. The elastomer is either one-component (EVF1) or two-component with one of the two parts containing the catalyst (EVF2, also referred to as RTV for “Room Temperature Vulcanization”). When the product is one-component, the polymerization reaction is activated by contact of the product with atmospheric moisture. When the product is two-component, crosslinking is not activated by atmospheric moisture. It starts when the two components are mixed together.

Japanese patent 82-40 558 describes the process for manufacturing a silicone elastomer matrix intended for diffusing fragrance into the atmosphere. The silicone elastomer composition is mixed with the fragrance, and crosslinking of the composition is then triggered in a mold by the addition of an organometallic salt. The matrix thus obtained has a nonlinear fragrancing diffusion due to the incompatibility of the polymer and the fragrance composition.

European patent EP 2 247 318 describes a transparent anhydrous gel in the form of a crosslinked silicone network, comprising volatile substance(s), notably fragrance, and nonvolatile substance(s), and not comprising any filler or reinforcing agent. Crosslinking of the silicone gel is obtained by the presence of a metal-type catalyst such as tin, titanium or platinum.

However, many of these devices have appreciable drawbacks. For example, the use of catalysts, notably metallic catalysts, makes these devices potentially hazardous to the environment due to their toxicity. Other devices require fillers or reinforcing agents, which have the drawback of making the gels opaque or increasing their hardness. Certain devices are opaque once formed, making them less attractive to consumers who want transparent products. For other types of devices, the problem will be related to the amount of volatile organic substance, which will be limited and thus less effective in space and time.

The biggest problem facing manufacturers is that silicone-type RTV polymer matrices obtained by mixing two different parts require a very long crosslinking time, when very little crosslinking agent is added, which is difficult to manage on an industrial scale. Today, manufacturers require a technology that allows them to control the crosslinking time of polymer matrices at will, and more particularly to accelerate said crosslinking time, while using a very small amount of crosslinking agent (which is necessary in order to obtain a gel that is not opaque and does not give off an unpleasant fish-like odor).

### SUMMARY

The present disclosure improves on the situation as presented above, while at the same time solving several technical problems.

Specifically, the Applicant has discovered that adding a very small amount of carboxylic acid to a two-part system for manufacturing an organic polymer matrix, also referred to hereinbelow as silicone gel, allowed the crosslinking time of said system to be controlled.

Thus, the present invention relates to a process for preparing a silicone gel which diffuses volatile organic substances, prepared using a two-component cold-vulcanizable silicone elastomer which does not include a metal catalyst, and whose crosslinking time can be controlled.

More precisely, a first subject of the present invention relates to a process for preparing a silicone gel which diffuses a volatile organic substance, said process comprising the following steps:
1) providing a part A comprising (i) from 10% to 89.99% by weight of a dihydroxy PDMS polymer having a viscosity measured at 25° C. of between 3000 and 100 000 cSt (mm²/s) and (ii) from 10% to 90% by weight of a mixture including a volatile solvent that is capable of dissolving the dihydroxy PDMS polymer and a volatile organic substance;
2) providing a part B comprising (iii) from 10% to 30% by weight of MMT and (iv) from 70% to 90% by weight of a non-functionalized PDMS silicone oil having a viscosity measured at 25° C. of between 10 and 245 cSt (mm²/s) that is capable of dissolving the MMT;
3) preparing a mixture comprising from 95% to 99% by weight of part A and from 1% to 5% by weight of part B;
characterized in that part A also comprises from 0.01% to 5% by weight of a carboxylic acid and does not comprise any metal catalyst.

A second subject of the present invention relates to a silicone gel that may be obtained via the process that is the subject of the present invention, characterized in that it does not comprise any metal catalyst.

A third subject of the present invention relates to the use of a carboxylic acid in a process for preparing a silicone gel which diffuses volatile organic substance from a two-component cold-vulcanizable silicone elastomer to control the crosslinking time of said elastomer.

A fourth subject of the present invention relates to a kit for performing the process according to the present invention, characterized in that it comprises:
a part A comprising (i) from 10% to 89.99% by weight of a dihydroxy PDMS polymer having a viscosity measured at 25° C. of between 3000 and 100 000 cSt (mm²/s) and (ii) from 10% to 90% by weight of a mixture including at least one volatile solvent that is capable of dissolving the dihydroxy PDMS polymer and a volatile organic substance;
a part B comprising (iii) from 10% to 30% by weight of MMT and (iv) from 70% to 90% by weight of a non-functionalized PDMS silicone oil having a viscosity measured at 25° C. of between 10 and 245 cSt (mm²/s) that is capable of dissolving the MMT;
characterized in that part A also comprises from 0.01% to 5% by weight of a carboxylic acid and does not comprise any metal catalyst and in that part A and part B are physically separated from each other.

### BRIEF DESCRIPTION OF THE DRAWINGS

Other features, details and advantages will emerge on reading the detailed description hereinbelow, and on analysis of the appended drawings, in which:

FIG. 1 shows the effect of adding myristic acid on the crosslinking time.

FIG. 2 shows the mole ratio of carboxylic acid/N-morpholinomethyl triethoxysilane (MMT).

### DETAILED DESCRIPTION OF EMBODIMENTS OF THE INVENTION

The silicone gel which diffuses volatile organic substances according to the present invention is a gel obtained from a two-component system that is crosslinkable at room temperature (RTV). Such systems are typically packaged in two distinct and physically separate parts, namely a part comprising a crosslinkable silicone elastomer (part A) and a crosslinking part (part B). The silicone gel is prepared by mixing the two parts together, and then allowing the gel to form, for example in a mold. The crosslinkable silicone compositions according to the present invention are functionalized silicone polymers.

According to the present invention, the silicone gel which diffuses volatile organic substances is prepared in the following manner:
1) providing a part A comprising (i) from 10% to 89.99% by weight of a dihydroxy PDMS polymer having a viscosity measured at 25° C. of between 3000 and 100 000 cSt (mm²/s) and (ii) from 10% to 90% by weight of a mixture including a volatile solvent that is capable of dissolving the dihydroxy PDMS polymer and a volatile organic substance;
2) providing a part B comprising (iii) from 10% to 30% by weight of MMT and (iv) from 70% to 90% by weight of a non-functionalized PDMS silicone oil having a viscosity measured at 25° C. of between 10 and 245 cSt (mm²/s) that is capable of dissolving the MMT;
3) preparing a mixture comprising from 95% to 99% by weight of part A and from 1% to 5% by weight of part B;
part A also comprising from 0.01% to 5% of a carboxylic acid and not comprising any metal catalyst.

The term “gel” means a homogeneous, elastic, network-forming composition having a relative resistance of less than 20 g/mm, in particular less than 15 g/mm, preferably less than 10 g/mm. The relative resistance of the gel is measured at 25° C. using a texturometer.

For example, the TA-XT Texturometer from the company Rheo may be used in “compressive force measurement” mode, using a 6 mm Stable Micro Systems SMS P/6 stainless steel cylindrical probe; test speed 0.50 mm/see; target mode: distance; break mode: rate; and break sensitivity 5.0 g.

### Part A

Part A comprises from 10% to 89.99% by weight of a dihydroxy PDMS polymer having a viscosity between 3000 and 100 000 cSt (mm²/s) (measured at 25° C.). In the context of the present invention, said functionalized silicone polymers are polydimethylsiloxanes functionalized at both ends with a hydroxyl group (also known as dihydroxy PDMS) having a viscosity of between 3000 and 100 000 cSt (mm²/s). Their viscosity measured at 25° C. may be between 3000 and 75 000 cSt (mm²/s), between 3000 and 50 000 cSt (mm²/s), between 3000 and 35 000 cSt (mm²/s), between 3000 and 10 000 cSt (mm²/s), between 3000 and 9000 cSt (mm²/s), between 4000 and 8000 cSt (mm²/s), between 5000 and 7000 cSt (mm²/s). Preferentially, the dihydroxy PDMS polymer has a viscosity measured at 25° C. equal to about 6000 cSt (mm²/s). The viscosity of the dihydroxy PDMS polymer, which is expressed as the kinematic viscosity in cSt (mm²/sec), is measured as dynamic viscosity (Pa-see units) at 25° C. using a TA Instruments Discovery HR-2 rheometer and the standard method provided by the standard DIN 53019 (2008) (including their calibration), and then converted from dynamic to kinematic viscosity by dividing the former by the density of the dihydroxy PDMS polymer.

The advantage of the present invention relative to existing systems is that said system does not require the addition of a metal catalyst to perform crosslinking, notably a tin catalyst.

Part A also comprises from 10% to 90% by weight of a mixture including a volatile solvent that is capable of dissolving the dihydroxy PDMS polymer and a volatile organic substance.

The term “volatile organic substance” means a product containing one or more organic molecules, which has a vapor pressure above atmospheric pressure at room temperature, i.e. 25° C. The volatile organic substance used according to the invention may be chosen from a fragrance, an odor-masking agent or an insecticide. Preferentially, it is a fragrance.

The fragrance may be chosen from a large number of odoriferous compounds. The fragrance according to the invention may be one or a combination of several odoriferous compounds. Such odoriferous compounds are mentioned, for example, in S. Arctander, “Perfume and Flavors” (Montclair, N.J., 1969), or in “Common Fragrance and Flavor Materials”, Wiley-VCH, Weinheim, 2006.

Nonlimiting examples of the latter are compounds belonging to the following families:
aromatic hydrocarbons, terpene and/or sesquiterpene, in particular essential oils containing these molecules and in particular essential oils of citrus fruits (lemon, orange, grapefruit, bergamot), nutmeg, etc.,
aromatic alcohols, in particular benzyl alcohol, phenylethyl alcohol and phenylpropyl alcohol,
alcohols and in particular cyclic or acyclic, saturated or unsaturated, primary, secondary or tertiary non-aromatic linalool, citronellol, geraniol, nerol, dihydromyrcenol, terpineol and fatty alicyclic alcohols containing from 4 to 10 carbon atoms in the chain,
aldehydes, notably saturated and unsaturated alicyclic fatty aldehydes with carbon chains containing 4 to 12 carbon atoms, aromatic aldehydes, such as cinnamaldehyde, alpha-amylcinnamaldehyde and alpha-hexylcinnamaldehyde aromatic aldehydes, lilial and phenolic aldehydes such as vanillin and ethylvanillin,
phenols and in particular aromatic phenols such as eugenol and isoeugenol, and also methyl ethers related thereto,
carboxylic acid esters, in particular acetic esters of benzyl alcohol, geraniol, citronellol, nerol, terpineol, borneol or linalool,
aromatic acid esters such as benzoates and salicylates, and also cinnamates esterified with alcohols of the aliphatic series containing a chain of 1 to 6 carbon atoms,
aromatic phenol acids, mainly in their aromatic lactone form, such as coumarin and dihydrocoumarin,
alcohol carboxylic acids in their lactone form, and dodeca-lactones, more particularly octa-, undeca- and gamma-dodeca-lactones, delta-deca-lactones, delta-undeca-lactones and delta-lactones in their saturated or unsaturated form,
macrocyclic compounds in which the carbon chain contains from 12 to 16 carbon atoms,
ethers and acetals in their acyclic or cyclic form, and in particular aromatic and non-aromatic aldehyde acetals containing a carbon chain of 4 to 10 carbon atoms, and also substituted furfuran cyclic ethers and substituted or unsubstituted pyran cyclic ethers,
heterocyclic compounds containing one nitrogen atom, and in particular indole derivatives, and also heterocyclic compounds containing two nitrogen atoms, and in particular those of the pyrazine series,
ketones, in particular aromatic ketones such as 4-(p-hydroxyphenyl)-2-butanone and cyclic or acyclic, saturated or unsaturated nonaromatic ketones, and in particular those of the pyrazine series,
aromatic or nonaromatic sulfides, disulfides and mercaptans.

The term “odor-masking agent” or “odor-neutralizing agent” means an agent that is capable of reducing or eliminating the perception of a bad odor generated by one or more molecules included in the composition of a product.

The odor-masking agent may be chosen from: a) monoesters; b) di- and/or tri-esters; c) alcohols, advantageously monoalcohols, comprising from 1 to 30 carbon atoms, said carbon atoms forming a linear or branched chain optionally including one or more unsaturations in the form of double bond(s), and optionally including a saturated or totally or partially unsaturated 5- or 6-membered ring structure; d) aldehydes and/or ketones, in particular aldehydes and/or ketones of formula R—CO—Rb, in which R represents a linear or branched hydrocarbon-based chain including from 1 to 6 carbon atoms, optionally comprising one or more unsaturations in the form of double bond(s), and R represents a hydrogen atom, a cyclic hydrocarbon-based chain or a linear or branched hydrocarbon-based chain, optionally, but preferably, substituted with a cyclic structure, R including from 6 to 12 carbon atoms, optionally comprising one or more unsaturations in the form of double bond(s) and optionally being substituted with one or more hydroxyl groups; and e) terpenes.

The term “insecticide” means an active substance or preparation which has the property of killing insects, in particular mosquitoes, or other invertebrates (mites, myriapods). The insecticide may be obtained by chemical synthesis or of plant origin. In the context of the present invention, the insecticide is of plant origin. It may be chosen from limonene or geraniol.

The concentration of the volatile organic substance in the volatile solvent may range from 0.05% to 50% by weight, in particular from 1% to 45% by weight, in particular from 2% to 40% by weight, in particular from 3% to 35% by weight, in particular from 5% to 30% by weight, or in particular from 10% to 45% by weight, in particular from 15% to 40% by weight, in particular from 20% to 35% by weight, relative to the total weight of part A.

The volatile solvent according to the invention may be chosen from a large number of solvents and may be one or a combination of several solvents. Preferably, the volatile solvent is odorless or only very sparingly odorous. In all cases, the volatile solvent must be capable of dissolving the dihydroxy PDMS.

In a first embodiment of the invention, the volatile solvent that is capable of dissolving dihydroxy PDMS is chosen from apolar solvents such as C7-C12 isoparaffins, the silicones hexamethyldisiloxane, octamethyltrisiloxane, decamethyltetrasiloxane, octamethyl-cyclotetrasiloxane, decamethylcyclopentasiloxane, docamethylcyclohexasiloxane and hexane. Commercial examples of volatile isoparaffins are Isopar C® to Isopar P® from the company Exxon Chemical, with a flash point from 40 to 100° C. Preferentially, the volatile solvent is C10-12 isoparaffin.

In a second embodiment of the invention, the volatile solvent that is capable of dissolving the dihydroxy PDMS is chosen from low-molecular-weight alkanes and sparingly polar solvents, such as fatty acid esters like isopropyl myristate, butyl myristate, isobutyl oleate, isopropyl oleate and diisopropyl adipate.

In addition, part A of the system comprises from 0.01% to 5% by weight of a carboxylic acid, in particular 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.50%, 1%, 1.50%, 2%, 2.50%, 3%, 3.50%, 4%, 4.50% or 5%.

The carboxylic acid allows the silicone elastomer's crosslinking time to be controlled by adjusting the amount of carboxylic acid used. The higher the amount of acid, the shorter the crosslinking time. The carboxylic acid thus allows the silicone elastomer crosslinking time to be accelerated.

The carboxylic acid is notably chosen from carboxylic acids of formula (I) below:

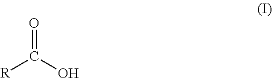

in which R represents hydrogen, linear or branched C1-C17 alkyl, linear or branched C5-C17 alkenyl, in particular branched C4-C5 alkenyl, or a phenyl group.

The carboxylic acid may be chosen from stearic acid, oleic acid, palmitic acid, myristic acid, lauric acid, undecylenic acid, capric acid, methyl-2-pentenoic acid, acetic acid, formic acid, isovaleric acid, methyl-2-butyric acid or phenylacetic acid.

In a preferred embodiment, the carboxylic acid is myristic acid.

In a preferred embodiment, the carboxylic acid is present at between 0.02% and 1% by weight relative to the weight of part A. In an even more preferred embodiment, the carboxylic acid is present at between 0.05% and 0.5% by weight relative to the weight of part A.

### Part B

Part B of the system comprises from 10% to 30% N-morpholinomethyl-triethoxysilane (MMT). This compound acts as a crosslinking agent within the system. This crosslinking agent is diluted in a solvent consisting of a non-functionalized PDMS silicone oil with a viscosity of between 10 and 245 cSt (mm²/s) (measured at 25° C.) which is capable of dissolving said MMT. The viscosity of the non-functionalized PDMS silicone oil, which is expressed as kinematic viscosity in cSt (mm²/sec), is measured as dynamic viscosity (Pa-sec units) at 25° C. using a TA Instruments Discovery HR-2 rheometer and the standard method provided by the standard DIN 53019 (2008) (including their calibration), and then converted from dynamic to kinematic viscosity by dividing the first by the density of the non-functionalized PDMS silicone oil. The solvent, for its part, is present in an amount of between 70% and 90% of part B.

Preferentially, the non-functionalized PDMS silicone oil has a viscosity measured at 25° C. of 10 cSt (mm²/s).

The Applicant found that there was an advantageous mole ratio between the amount of carboxylic acid and the amount of MMT used in the process of the present invention. This advantageous mole ratio between the amount of carboxylic acid and the amount of MMT is between 0.1 and 1. In a particular embodiment of the invention, the ratio between the amount of carboxylic acid and the amount of MMT is between 0.1 and 1. A ratio greater than 0.1 makes it possible to obtain good crosslinking times, i.e. under 2 hours, without using too much carboxylic acid. When the ratio is above 1, the addition of carboxylic acid has less effect on the crosslinking time.

A second subject relates to a silicone gel which can be obtained via the process that is the subject of the present invention, characterized in that it does not comprise any metal catalyst.

The gel according to the invention is characterized in that it allows diffusion, i.e. controlled release in time and space of the volatile organic substance. Thus, the gel can be used to fragrance or deodorize the atmosphere, linen, or as an insect repellent or insecticide. Preferably, the gel is used for fragrancing the atmosphere. When used as a fragrancing or deodorizing product, the silicone gel may be used in any type of environment, notably in domestic, commercial or industrial premises or in a car.

The silicone gel according to the present invention is characterized in that it has the advantage of being transparent.

The term “transparent” means that the gel has a haze of less than 40 NTU (nephelometric turbidity units) at 25° C., measured using a turbidimeter. The term “transparent” according to the present invention does not mean “colorless”. Thus, a transparent gel may be a transparent and colored gel.

In a preferred embodiment of the invention, the gel haze is less than 20 NTU, more preferably less than 12 NTU, most preferably less than 8 NTU. NTU (nephelometric turbidity units) is the unit used to describe haze. The greater the diffusion, the higher the haze. Consequently, low NTU values indicate high clarity, while high NTU values indicate low clarity. The turbidimeter measurement process is based on a comparison of the intensity of light scattered by the sample under defined conditions with the intensity of light scattered by a standard reference suspension, which is normally haze-free water (distilled water or distilled water passed through a 0.45 μm membrane filter). The turbidimeter will consist of a nephelometer with a light source allowing the sample to be illuminated, and one or more photoelectric detectors with a reading device to indicate the intensity of light scattered at right angles to the incident light path. The turbidimeter must be designed so that little stray light reaches the detector in the absence of haze, and must be free of significant drift after a short heating period.

The turbidimeter models 2100 and 2100 A, from the Hach company, are widely used and have proven to be reliable; however, other instruments meeting the above design criteria are acceptable, for example the Turbi-direct® machine sold by the company Aqua lytique.

Advantageously, the silicone gel according to the invention does not comprise any reinforcing agent.

Advantageously, silicone gel according to the invention has a relative resistance of less than 20 g/mm.

In addition, the gel according to the invention is anhydrous. The term “anhydrous” means “water-free”, i.e. a gel in which no water is present in the form of hydrate or water of crystallization.

A third subject of the present invention relates to the use of a carboxylic acid in a process for preparing a silicone gel which diffuses a volatile organic substance from a two-component cold-vulcanizable silicone elastomer to control the crosslinking time of said elastomer.

Not only does the carboxylic acid allow the crosslinking time to be controlled, but more particularly it allows the crosslinking time of said elastomer to be accelerated in a controlled manner.

The carboxylic acid that may be used in the present invention has the general formula (I) as follows

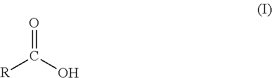

in which R represents hydrogen, linear or branched C1-C17 alkyl, linear or branched C5-C17 alkenyl, in particular branched C4-C5 alkenyl, or a phenyl group.

In a preferred embodiment, the carboxylic acid used is chosen from stearic acid, oleic acid, palmitic acid, myristic acid, lauric acid, undecylenic acid, capric acid, methyl-2-pentenoic acid, acetic acid, formic acid, isovaleric acid, methyl-2-butyric acid and phenylacetic acid. Preferentially, the carboxylic acid is myristic acid.

A fourth subject of the present invention relates to a kit for performing the process according to the present invention, characterized in that it comprises:
a part A comprising (i) from 10% to 89.99% by weight of a dihydroxy PDMS polymer having a viscosity measured at 25° C. of between 3000 and 100 000 cSt (mm²/s) and (ii) from 10% to 90% by weight of a mixture including at least one volatile solvent that is capable of dissolving the dihydroxy PDMS polymer and a volatile organic substance;
a part B comprising (iii) from 10% to 30% by weight of MMT and (iv) from 70% to 90% by weight of a non-functionalized PDMS silicone oil having a viscosity measured at 25° C. of between 10 and 245 cSt (mm²/s) that is capable of dissolving the MMT;
characterized in that part A also comprises from 0.01% to 5% by weight of a carboxylic acid and does not comprise any metal catalyst and in that part A and part B are physically separated from each other.

In the kit, parts A and B are as defined previously.

The invention is illustrated below by the following examples, which should not be considered as limiting the scope of the invention, and should be read with reference to the figures.

### EXAMPLES

### Example 1: Composition of Part A (Silicone Matrix)

Dihydroxy PDMS (polymer purchased from Wacker®, trade name FD6 6000 cSt

$$( {mm}^{2} / s ) ) = 60 g ( 60 \% by weight )$$

$$Fragrance = 30 g ( 30 \% by weight )$$

$$Isopar L ( Exxon ) = 4.6 g ( 4.6 \% by weight )$$

$$Isopar M ( Exxon ) = 5 g ( 5 \% by weight )$$

$$Myristic acid ( MA ) = 0.4 g ( 0.4 \% by weight )$$

The preferred myristic acid percentage is 0.4% by weight, but this amount may change as a function of the fragrance used and the desired crosslinking time.

### Example 2: Composition of Part B (Crosslinking Agent)

$$Silicone oil 10 cSt ( {mm}^{2} / s ) = 85 \% by weight$$

$$MMT ( ( N - morpholinomethyl ) triethoxysilane ) CAS \# 21743 - 27 - 1 = 15 \% by weight$$

This crosslinking agent solution is purchased from Wacker® under the name HC1015 and is used as such.

### Example 3: Fragranced Silicone Gel

$$Part A according to Example 1 = 97 \% by weight$$

$$Part B according to Example 2 = 3 \% by weight$$

Add Part B (crosslinking agent) to Part A and mix until a homogeneous mixture is obtained. Pour the mixture into a glass or PET container, close and allow to gel.

### Example 4: Action of Various Acids

To demonstrate the similar effect of different carboxylic acids, several silicone gels were prepared with the same molar amount of acid.

The gels were prepared according to the formula and process indicated in Example 3. To prepare these gels, the fragrance described in Example 1 was replaced with an equivalent amount of Isopar L.

About 1 g of this mixture is then placed in the rheometer to perform the crosslinking time measurement.

### Procedure for Measuring the Crosslinking Time

The crosslinking time is measured using a TA-Instruments Discovery HR-2 rheometer. The measurement is made in Oscillation-Time mode, and the crosslinking time is taken at the intersection of the elastic modulus and the G′ and G″ loss moduli.

The rheometer measurement protocol is described below:
Geometry: 40.0 mm 3.9875° cone-plate
Mode: Oscillation-Time
Temperature: 25° C.
Sampling interval: 30.0 s/pt
Stress: 1%
Frequency: 1 Hz
Gap: 110 μm

| Part A = 97% | | | | | |
|---|---|---|---|---|---|
| | | | % Isopar | | Cross- |
| % Polymer | | % Acid | L | Part B | linking |
| (% by | | (% by | (% by | (% by | time |
| weight) | Acid used | weight) | weight) | weight) | (min) |
| 60% | C18 Stearic acid | 0.2% | 39.8% | 3% | 13 |
| 60% | C14 Myristic acid | 0.16% | 39.84% | 3% | 9 |
| 60% | C16 Palmitic acid | 0.18% | 39.82% | 3% | 11 |
| 60% | C12 Lauric acid | 0.14% | 39.86% | 3% | 9 |
| 60% | C18 Oleic acid | 0.19% | 39.81% | 3% | 13 |
| 60% | C10 Capric acid | 0.12% | 39.88% | 3% | 13 |
| 60% | C11 Undecylenic | 0.13% | 39.87% | 3% | 9 |
| | acid | | | | |
| 60% | C2 Acetic acid | 0.04% | 39.96% | 3% | 10 |
| 60% | C1 Formic acid | 0.03% | 39.97% | 3% | 6 |
| 60% | Phenylacetic acid | 0.08% | 39.92% | 3% | 3 |
| 60% | Isovaleric acid | 0.07% | 39.93% | 3% | 11 |
| 60% | C05 Methyl-2- | 0.07% | 39.93% | 3% | 12 |
| | butyric acid | | | | |

This example highlights the fact that various carboxylic acids of different chain lengths may be used to reduce the crosslinking time.

Furthermore, it is found that the chain length of the carboxylic acid used has no influence on the crosslinking time.

### Example 5: Controlling the Crosslinking Time

To highlight the possibility of controlling the crosslinking time by addition of a very small amount of carboxylic acid, several gels were prepared with different amounts of myristic acid.

The gels were prepared according to the formula and process indicated in Example 3. To prepare these gels, the fragrance described in Example 1 was replaced with an equivalent amount of Isopar L.

About 1 g of this mixture was then placed in the rheometer to perform the crosslinking time measurement as described previously.

To highlight the possibility of controlling the crosslinking time by adding a very small amount of myristic acid.

TABLE 2
Effect of myristic acid percentage on the crosslinking time

| | | | Part B 3% by | |
|---|---|---|---|---|
| | | | weight | |
| Part A = 97% by weight | | | Silicone oil 10 | |
| | | % Myristic | cSt (mm²/s) | Cross- |
| % Polymer | % Isopar L | acid | 85% by weight/ | linking |
| (% by | (% by | (% by | MMT 15% by | time in |
| weight) | weight) | weight) | weight mixture | minutes |
| 60% | 40% | 0% | 3% | 277 |
| 60% | 39.996% | 0.016% | 3% | 80 |
| 60% | 39.96% | 0.04% | 3% | 34 |
| 60% | 39.8% | 0.2% | 3% | 11 |
| 60% | 39.6% | 0.4% | 3% | 5 |

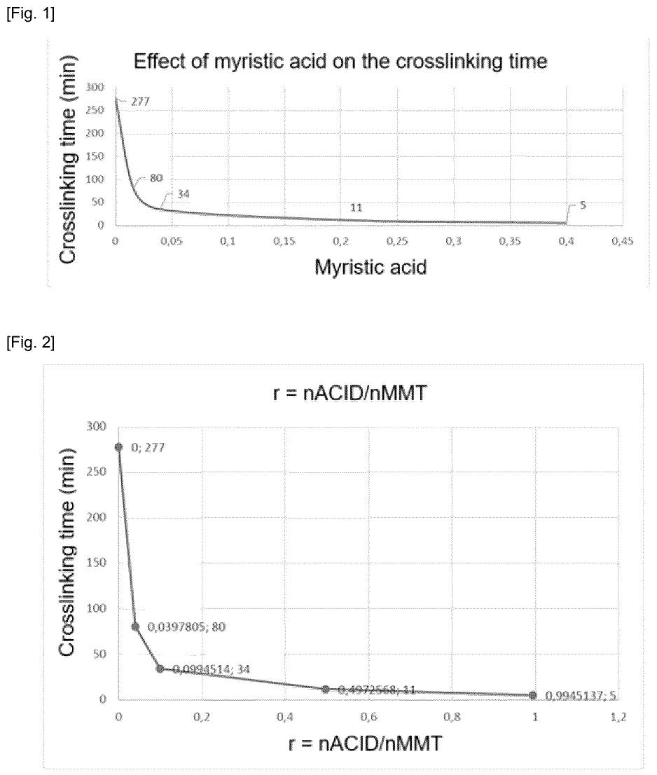

As shown in Table 2 and also in FIG. 1, the addition of a very small amount of myristic acid allows the crosslinking time of the silicone matrix+crosslinking agent mixture (Part A+Part B) to be reduced. The higher the amount of acid, the shorter the crosslinking time. The user can thus control the crosslinking time by adjusting the amount of acid (in this case myristic acid) added.

FIG. 2 represents the mole ratio (r)=n acid/n MMT. The graph represented in FIG. 2 is advantageous when it is desired to know what amount of a given carboxylic acid must be used to obtain the desired crosslinking time.

### Example 6: Crosslinking Time for Fragranced Formulations

To highlight the fact that the crosslinking time can be controlled when using different fragrance compositions, several gels were prepared according to the formula given in Example 3.

TABLE 3
crosslinking times obtained when preparing silicone gels made with different fragrance compositions.

| Part A = 97% by weight | | | | | Part B = | Crosslinking |
|---|---|---|---|---|---|---|
| % polymer | Fragrance | % Fragrance | % Isopar L | % Myristic acid | 3% by | time in |
| (% by weight) | reference | (% by weight) | (% by weight) | (% by weight) | weight | minutes |
| 60% | Mint perfume | 30% | 10% | 0% | 3% | 159 |
| 60% | Mint perfume | 30% | 9.6% | 0.4% | 3% | 41 |
| 60% | Black energy perfume | 30% | 10% | 0% | 3% | 226 |
| 60% | Black energy perfume | 30% | 9.6% | 0.4% | 3% | 57 |
| 60% | Oceanic perfume | 30% | 10% | 0% | 3% | 208 |
| 60% | Oceanic perfume | 30% | 9.6% | 0.4% | 3% | 117 |
| 60% | Peach perfume | 30% | 10% | 0% | 3% | 340 |
| 60% | Peach perfume | 30% | 9.6% | 0.4% | 3% | 102 |
| 60% | Lavender perfume | 30% | 10% | 0% | 3% | 77 |
| 60% | Lavender perfume | 30% | 9.6% | 0.4% | 3% | 37 |
| 60% | Lavender perfume | 30% | 9.2% | 0.8% | 3% | 26 |
| | Fragrance-free gel | 0% | 40% | 0% | 3% | 277 |

As this table shows, the addition of carboxylic acid allows the crosslinking time of the fragranced gels described in Table 3 to be significantly reduced.

1. A process for preparing a silicone gel which diffuses a volatile organic substance, said process comprising the following steps:
1) providing a part A comprising (i) from 10% to 89.99% by weight of a dihydroxy polydimethylsiloxane polymer having a viscosity measured at 25° C. of between 3000 and 100 000 mm²/s and (ii) from 10% to 90% by weight of a mixture including:
a volatile solvent that is capable of dissolving the dihydroxy polydimethylsiloxane polymer, and
a volatile organic substance;
2) providing a part B comprising (iii) from 10% to 30% by weight of N-morpholinomethyl triethoxysilane and (iv) from 70% to 90% by weight of a non-functionalized polydimethylsiloxane silicone oil having a viscosity measured at 25° C. of between 10 and 245 mm2/s which is capable of dissolving N-morpholinomethyl triethoxysilane;
3) preparing a mixture comprising from 95% to 99% by weight of part A and from 1% to 5% by weight of part B;
characterized in that part A also comprises from 0.01% to 5% by weight of a carboxylic acid and does not comprise any metal catalyst.

2. The process as claimed in claim 1, characterized in that the carboxylic acid has the general formula (I) below: 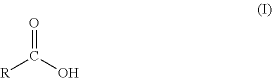
in which R represents hydrogen, linear or branched C1-C17 alkyl, linear or branched C5-C17 alkenyl, in particular branched C4-C5 alkenyl, or a phenyl group.

3. The process as claimed in claim 1, characterized in that the carboxylic acid is chosen from stearic acid, oleic acid, palmitic acid, myristic acid, lauric acid, undecylenic acid, capric acid, methyl-2-pentenoic acid, acetic acid, formic acid, isovaleric acid, methyl-2-butyric acid or phenylacetic acid.

4. The process as claimed in claim 1, characterized in that the carboxylic acid is myristic acid.

5. The process as claimed in claim 1, characterized in that the carboxylic acid is present at between 0.02% and 1% by weight relative to the total weight of part A.

6. The process as claimed in claim 1, characterized in that the dihydroxy polydimethylsiloxane has a viscosity measured at 25° C. of between 4000 and 8000 mm2/s.

7. The process as claimed in claim 1, characterized in that the volatile organic substance is chosen from a fragrance, an odor-masking agent or an insecticide.

8. The process as claimed in claim 1, characterized in that the concentration of the volatile organic substance in the volatile solvent is from 0.05% to 50% by weight, in particular from 5% to 30% by weight relative to the total weight of part A.

9. The process as claimed in claim 1, characterized in that the volatile solvent that is capable of dissolving the dihydroxy polydimethylsiloxane is chosen from apolar solvents, such as C7-C12 isoparaffins, the silicones hexamethyldisiloxane, octamethyltrisiloxane, decamethyltetrasiloxane, octamethylcyclotetrasiloxane, decamethylcyclopentasiloxane, docamethylcyclohexasiloxane, and hexane.

10. The process as claimed in claim 1, characterized in that the volatile solvent that is capable of dissolving the dihydroxy polydimethylsiloxane is chosen from low molecular weight alkanes and sparingly polar solvents, such as fatty acid esters like isopropyl myristate, butyl myristate, isobutyl oleate, isopropyl oleate, and diisopropyl adipate.

11. The process as claimed in claim 9, characterized in that the volatile solvent is C10-C12 isoparaffin.

12. The process as claimed in claim 1, characterized in that the mole ratio between the amount of carboxylic acid and the amount of N-morpholinomethyl triethoxysilane is between 0.1 and 1.

13. A silicone gel which diffuses a volatile organic substance, which may be obtained via the process as claimed in claim 1, characterized in that it does not comprise any metal catalyst.

14. The silicone gel which diffuses a volatile organic substance as claimed in claim 13, characterized in that it is transparent.

15. The silicone gel which diffuses a volatile organic substance as claimed in claim 13, characterized in that it does not comprise any reinforcing agent.

16. The silicone gel which diffuses a volatile organic substance as claimed in claim 13, characterized in that it has a relative resistance of less than 20 g/mm.

17. Use of a carboxylic acid in a process for preparing a silicone gel which diffuses volatile organic substance from a two-component cold-vulcanizable silicone elastomer to control the crosslinking time of said elastomer.

18. The use as claimed in claim 17, for accelerating the crosslinking time of said elastomer in a controlled manner.

19. The use as claimed in claim 17, characterized in that the carboxylic acid has the general formula (I) below: 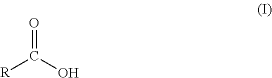
in which R represents hydrogen, linear or branched C1-C17 alkyl, linear or branched C5-C17 alkenyl, in particular branched C4-C5 alkenyl, or a phenyl group.

20. The use as claimed in claim 17, characterized in that the carboxylic acid is chosen from stearic acid, oleic acid, palmitic acid, myristic acid, lauric acid, undecylenic acid, capric acid, methyl-2-pentenoic acid, acetic acid, formic acid, isovaleric acid, methyl-2-butyric acid or phenylacetic acid.

21. The use as claimed in claim 17, characterized in that the carboxylic acid is myristic acid.

22. A kit for performing the process as claimed in claim 1, said kit comprising:
a part A comprising (i) from 10% to 89.99% by weight of a dihydroxy polydimethylsiloxane polymer having a viscosity measured at 25° C. of between 3000 and 100 000 mm2/s and (ii) from 10% to 90% by weight of a mixture including at least one volatile solvent that is capable of dissolving the dihydroxy polydimethylsiloxane polymer and a volatile organic substance;
a part B comprising (iii) from 10% to 30% by weight of N-morpholinomethyl triethoxysilane and (iv) from 70% to 90% by weight of a non-functionalized polydimethylsiloxane silicone oil having a viscosity measured at 25° C. of between 10 and 245 mm2/s which is capable of dissolving the N-morpholinomethyl triethoxysilane;
characterized in that part A also comprises from 0.01% to 5% by weight of a carboxylic acid and does not comprise any metal catalyst and in that part A and part B are physically separated from each other.
